(19) United States
(12) Patent Application Publication
(10) Pub. No.: US 2019/0290365 A1
(43) Pub. Date: Sep. 26, 2019

(54) METHOD AND APPARATUS FOR PERFORMING IMAGE GUIDED MEDICAL PROCEDURE

(71) Applicant: Fei Gao, Buena Park, CA (US)

(72) Inventor: Fei Gao, Buena Park, CA (US)

(73) Assignee: GuideMia Technologies Inc., Los Alamitos, CA (US)

(21) Appl. No.: 15/936,373

(22) Filed: Mar. 26, 2018

(51) Int. Cl. A61B 34/20; A61B 90/00; A61B 6/14; A61C 13/34; A61C 13/00; A61C 1/08; A61B 6/03; A61B 5/055; A61B 34/10

(52) U.S. Cl. CPC A61B 34/20; A61B 2034/2065; A61B 6/14; A61C 13/34; A61C 13/0004; A61C 1/082; A61B 6/032; A61B 5/055; A61B 34/10; A61B 2090/3983; A61B 2090/374; A61B 2090/3762; A61B 2090/3937; A61B 2034/105; A61B 2090/364; A61B 2034/2051; A61B 90/39

(57) ABSTRACT

The present invention provides a method and an apparatus for performing image guided medical procedure. In generating a virtual anatomical part such as a virtual jawbone for treatment planning, imaging techniques such as CT or MRI scanning of the actual jawbone is accomplished with no actual tracking marker attached to the patient, or with no virtual model of actual tracking markers being acquired and subsequently used in any other step of the method.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### FIELD OF THE INVENTION

The present invention generally relates to a stereotactic medical procedure performed on an anatomy of a patient, and a system used for the procedure. Although the invention will be illustrated, explained and exemplified by a surgical navigation system and an image guided procedure that tracks both a portion of a patient's anatomy such as jaw bone and an instrument such as a dental drill, relative to a navigation base such as image data, it should be appreciated that the present invention can also be applied to other fields, for example, physiological monitoring, guiding the delivery of a medical therapy, and guiding the delivery of a medical device, an orthopedic implant, or a soft tissue implant in an internal body space.

### BACKGROUND OF THE INVENTION

Stereotactic surgery is a minimally invasive form of surgical intervention, in which a three-dimensional coordinate system is used to locate targets inside the patient's body and to perform some action on them such as drilling, ablation, biopsy, lesion, injection, stimulation, implantation, and radiosurgery (SRS). Plain X-ray images (radiographic mammography), computed tomography (CT), and magnetic resonance imaging (MRI) can be used to guide the procedure. Stereotactic surgery works on the basis of three main components. (1) a computer based stereotactic planning system, including atlas, multimodality image matching tools, coordinates calculator, etc.; (2) a stereotactic device or apparatus; and (3) a stereotactic localization and placement procedure.

For example, in an image-guided surgery, the surgeon utilizes tracked surgical instruments in conjunction with preoperative or intraoperative images in order to indirectly guide the procedure. Image guided surgery systems use cameras or electromagnetic fields to capture and relay the patient's anatomy and the surgeon's precise movements of the instrument in relation to the patient, to computer monitor in the operating room.

Real time image guided surgery has been introduced into dental and orthopedic area for years. Typically, a system includes a treatment planning software, a marker system attached to patient's anatomy, a 3D camera system to track the markers, a registration software module to align the actual patient position with the patient image in the treatment plan, a software module to display the actual surgical tool positions and the planned positions on the computer screen.

The most important part of the system is the fiducial markers and the marker tracking system. In principle, the fiducial markers must be placed onto the patient's anatomy before surgery and during the surgery. The relative positons between the markers and the surgery site must be fixed. For example, in a dental implant placement system, if the doctor is going to place implants on the lower jaw, the markers have to be placed on the lower jaw, and they shall not move in the process. If the markers are placed onto for example the upper jaw, they would be useless because the jaws can move relative to each other all the time.

For example, with current dental implant navigation systems, as well as other surgical navigation systems, the procedure is pretty much standard, and all have a fiducial marker or markers attached to the surgical site before the data acquisition and during the surgery.

Typically, before the surgery, a stent or a clip is made to be fit onto the patient's teeth, and then some fiducial markers are attached to the stent. A CT scan is performed with the stent in the patient's mouth. In the CT scan, the markers will be recognized and their relationships with the patient's bone structure will be identified. Then the stent and markers are removed from the patient's mouth, and then installed back before the surgery. The navigation system will then identify the markers during the surgery and dynamically register them with the markers in the pre-op image data, and therefore the computer software can find out the position of the patient bone structure during the entire surgery.

However, the approach has very obvious drawbacks. The stent or clip has to be customized to patient's teeth or other dental structure so that it can be repositioned to exact same position before scan and before surgery. For edentulous cases, the approach needs special handling because the placement of the stent of the soft tissue is very inaccurate. Even with existing teeth, it can introduce positioning error to clip the stent over the teeth before data acquisition, remove it after acquisition, and the clip it back on before surgery. Moreover, the size of the stent is very crucial to the procedure too. If it is too small, repositioning the stent can be inaccurate. Practically the patient has to be CT scanned in the doctor's office unless the stent goes with the patient to other facility.

Therefore, there exists a need to overcome the aforementioned problems. Advantageously, the present invention provides a method and an apparatus for performing image guided medical procedure which exhibits numerous technical merits. For example, the image guided surgery can be performed without pre-attached markers such as fiducial markers being involved in data preparation. The patient's actual anatomical information is obtained during the surgery preparation, and is used to register tracking markers and patient's anatomy.

### SUMMARY OF THE INVENTION

One aspect of the present invention provides a method of performing an image guided medical procedure. The method includes the following steps: (1) providing an actual anatomical part of a patient, (2) generating a virtual anatomical part for treatment planning from at least CT or MRI scanning of the actual anatomical part; (3) attaching actual tracking markers to the actual anatomical part, wherein the actual anatomical part and the actual tracking markers attached therewith maintain a spatial relationship during the image guided medical procedure; (4) acquiring a virtual combined model of the actual tracking markers and at least a part of the actual anatomical part, wherein the virtual combined model comprises a first sub-model from the actual tracking markers and a second sub-model from the at least a part of the actual anatomical part; (5) registering the virtual combined model to the virtual anatomical part by selecting at least a part of the second sub-model and matching the part to its counterpart in the virtual anatomical part; (6) generating a working model including the virtual anatomical part and virtual tracking markers, the two having a spatial relationship same as the spatial relationship in step (3); and (7) during the rest of the image guided medical procedure, tracking position and orientation of the actual tracking markers, registering the tracked position and orientation of the actual tracking markers to the working model, and calculating and tracking position and orientation of the virtual anatomical part in real-time based on the spatial relationship in step (6) which are the same as the position and orientation of the actual anatomical part.

Another aspect of the invention provides an apparatus for performing an image guided medical procedure. The apparatus includes the following components: (1) a first module (or control circuit) for generating a virtual anatomical part for treatment planning from at least CT or MRI scanning of an actual anatomical part of a patient; (2) a second module (or control circuit) for acquiring a virtual combined model of actual tracking markers and at least a part of an actual anatomical part, wherein the virtual combined model comprises a first sub-model from the actual tracking markers and a second sub-model from said at least a part of the actual anatomical part, wherein the actual tracking markers are attached to the actual anatomical part; and wherein the actual anatomical part and the actual tracking markers attached therewith maintain a spatial relationship during the image guided medical procedure; (3) a third module (or control circuit) for registering the virtual combined model to said virtual anatomical part by selecting at least a part of the second sub-model and matching the part to its counterpart in the virtual anatomical part, (4) a fourth module (or control circuit) for generating a working model including the virtual anatomical part and virtual tracking markers, the two having a spatial relationship same as the spatial relationship in the second module; and (5) a tracking system for tracking position and orientation of the actual tracking markers, registering the tracked position and orientation of the actual tracking markers to the working model, and calculating and tracking position and orientation of the virtual anatomical part in real-time based on the spatial relationship in the fourth module, which are the same as the position and orientation of the actual anatomical part, during the image guided medical procedure.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 is a block diagram of a method of performing an image guided medical procedure in accordance with an exemplary embodiment of the present invention.

FIG. 2 schematically shows CT or MRI scanning of an actual anatomical part (AAP) without pre-attached markers in accordance with an exemplary embodiment of the present invention.

FIG. 3 illustrates attachment of actual tracking markers (ATMs) to an actual anatomical part (AAP) in accordance with an exemplary embodiment of the present invention.

FIG. 4 demonstrates acquisition of a virtual combined model of the ATMs and at least a part of AAP in accordance with an exemplary embodiment of the present invention.

FIG. 5 depicts the registration of a virtual combined model to a virtual anatomical part (VAP) in accordance with an exemplary embodiment of the present invention

FIG. 6 schematically shows a medical procedure with the capability of tracking position and orientation of actual anatomical part (AAP) in accordance with an exemplary embodiment of the present invention.

FIG. 7 is a block diagram of step (4) showing acquisition of a virtual combined model of the actual tracking markers and at least a part of the actual anatomical part in accordance with an exemplary embodiment of the present invention.

FIG. 8 is a block diagram of a step of providing a probe in accordance with an exemplary embodiment of the present invention.

FIG. 9A schematically illustrates the calibration of a probe and the use of the calibrated probe in accordance with an exemplary embodiment of the present invention,

FIG. 9B schematically illustrates the collection of the individual datasets using the calibrated probe in accordance with an exemplary embodiment of the present invention.

FIG. 10 is another block diagram with illustration of step (4) showing acquisition of a virtual combined model of the actual tracking markers and at least a part of the actual anatomical part in accordance with an exemplary embodiment of the present invention.

FIG. 11 schematically depicts an apparatus for performing the image guided medical procedure in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

It is also to be understood that the terminology used herein is for the purpose of describing particular embodiments only, and is not intended to limit the scope of the invention. For example, when an element is referred to as being “on”, “connected to”, or “coupled to” another element, it can be directly on, connected or coupled to the other element or intervening elements may be present. In contrast, when an element is referred to as being “directly on”, “directly connected to”, or “directly coupled to” another element, there are no intervening elements present.

Image registration is the process of transforming different sets of data into one coordinate system. Data may be multiple photographs, data from different sensors, times, depths, or viewpoints. It is used in computer vision, medical imaging, military automatic target recognition, and compiling and analyzing images and data from satellites. Registration is necessary in order to be able to compare or integrate the data obtained from these different measurements.

The terms “registration”, “matching” and “alignment” used in some embodiments of the present invention should be appreciated in the context of the following description. Image registration or image alignment algorithms can be classified into intensity-based and feature-based. One of the images is referred, to as the reference or source and the others are referred to as the target, sensed or subject images. Image registration involves spatially transforming the source/reference image(s) to align with the target image. The reference frame in the target image is stationary, while the other datasets are transformed to match to the target. Intensity-based methods compare intensity patterns in images via correlation metrics, while feature-based methods find correspondence between image features such as points, lines, and contours. Intensity-based methods register entire images or sub-images. If sub-images are registered, centers of corresponding sub-images are treated as corresponding feature points. Feature-based methods establish a correspondence between some especially distinct points in images. Knowing the correspondence between the points in images, a geometrical transformation is then determined to map the target image to the reference images, thereby establishing point-by-point correspondence between the reference and target images.

In computer vision and pattern recognition, point set registration, also known as point matching, is the process of finding a spatial transformation that aligns two point sets. The purpose of finding such a transformation includes merging multiple data sets into a globally consistent model, and mapping a new measurement to a known data set to identify features or to estimate its pose. A point set may be raw data from 3D scanning or an array of rangefinders. For use in image processing and feature-based image registration, a point set may be a set of features obtained by feature extraction from an image, for example corner detection. Point set registration is used in optical character recognition, augmented reality and aligning data from magnetic resonance imaging with computer aided tomography scans.

Given two point sets, rigid registration yields a rigid transformation which maps one point set to the other. A rigid transformation is defined as a transformation that does not change the distance between any two points. Typically such a transformation consists of translation and rotation. Sometimes, the point set may also be mirrored. Iterative Closest Point (ICP) is an algorithm employed to minimize the difference between two clouds of points. ICP is often used to reconstruct 2D or 3D surfaces from different scans, to localize robots and achieve optimal path planning (especially when wheel odometry is unreliable due to slippery terrain), to co-register bone models, etc. In the Iterative Closest Point or, in some sources, the Iterative Corresponding Point, one point cloud (vertex cloud), the reference, or target, is kept fixed, while the other one, the source, is transformed to best match the reference. The algorithm iteratively revises the transformation (combination of translation and rotation) needed to minimize an error metric, usually a distance from the source to the reference point cloud, such as the sum of squared differences between the coordinates of the matched pairs. ICP is one of the widely used algorithms in aligning three dimensional models given an initial guess of the rigid body transformation required. The inputs may be reference and source point clouds, initial estimation of the transformation to align the source to the reference (optional), criteria for stopping the iterations. The output may be refined transformation. Essentially, the algorithm steps include (1) for each point (from the whole set of vertices usually referred to as dense or a selection of pairs of vertices from each model) in the source point cloud, matching the closest point in the reference point cloud (or a selected set); (2) estimating the combination of rotation and translation using a root mean square point to point distance metric minimization technique which will best align each source point to its match found in the previous step after weighting and rejecting outlier points; (3) transforming the source points using the obtained transformation; (4) iterating (re-associating the points, and so on). There many ICP variants such as point-to-point and point-to-plane.

The terms “single-modality” and “multi-modality” are defined as that single-modality methods tend to register images in the same modality acquired by the same scanner/sensor type, while multi-modality registration methods tended to register images acquired by different scanner/sensor types. Multi-modality registration methods are preferably used in the medical imaging of the invention, as images of a patient are frequently obtained from different scanners. Examples include registration of brain CT/MRI images or whole body PET/CT images for tumor localization, registration of contrast-enhanced CT images against non-contrast-enhanced CT images for segmentation of specific parts (such as teeth) of the anatomy, and registration of ultrasound and CT images for prostate localization in radiotherapy.

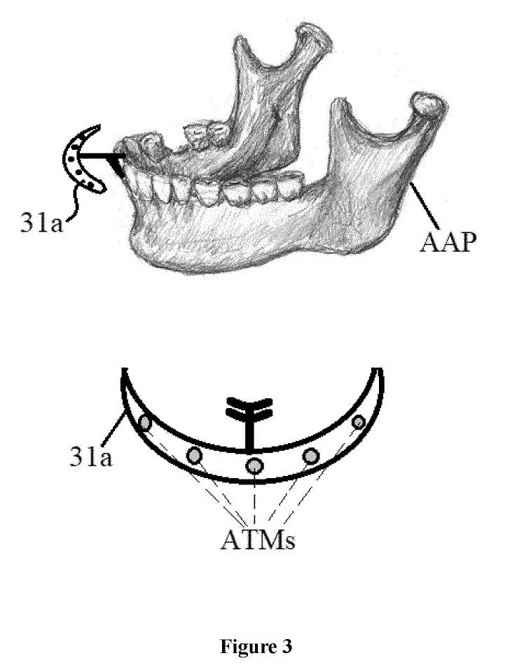

A best mode embodiment of the invention may be a treatment planning and surgical procedure as describe in the following. First, a CT scan of the patient, or other imagery modality, is acquired. No markers need to be attached to the patient's dental structure. The CT data is loaded into a software system for treatment planning. At the surgery time, a small positioning device (e.g. 31a in FIG. 3) is attached to one or more teeth, to a bone, or to soft tissue, whatever is applicable The device can be of any shape and size as long as it can be secured. The device will have a plurality of tracking markers. The markers can be of any kind, such as geometric shapes, geometry patterns, passive IR markers, or active markers emitting electromagnetic signals. In one embodiment, the positioning device can be a pre-made model with markers. The model can be secured onto patient's teeth or tissue with any mechanism. For example, it can simply have an adjustable screw. The patient is then placed into the field of view of a navigation tracking device. A special procedure and corresponding software and hardware are employed to register the actual patient position and the patient image data in the software system, and also register the tracking/fiducial markers on the positioning device with the patient data. The invention can perform such registration without using the positioning device in the initial CT scan. The surgical handpiece and drill bits or any other components of the surgical tools that will perform the surgical operations are registered with the patient data. During the surgical operations, the tracking system and software will work together to track the tool positions related to the patient's surgical site, and guide the doctor to finish the surgery with graphical and image feedback, or any other computer-human interface such as voice guidance.

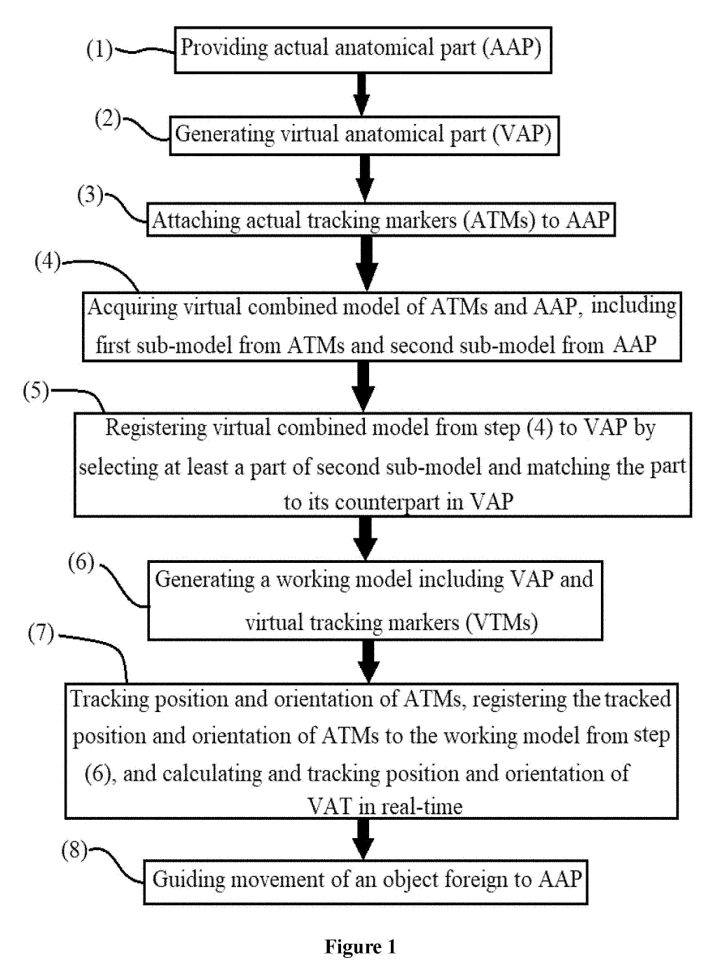

Embodiments more general than the best mode embodiment as described above can be illustrated in FIGS. 1-11. Referring to FIG. 1, an exemplary method of performing an image guided medical procedure is illustrated. Step (1) of the method is providing an actual anatomical part (AAP) of a patient P such as teeth, jawbone, brain, and skull. Step (2) is generating a virtual anatomical part (VAP) for treatment planning from at least CT or MRI scanning of the actual anatomical part (AAP).

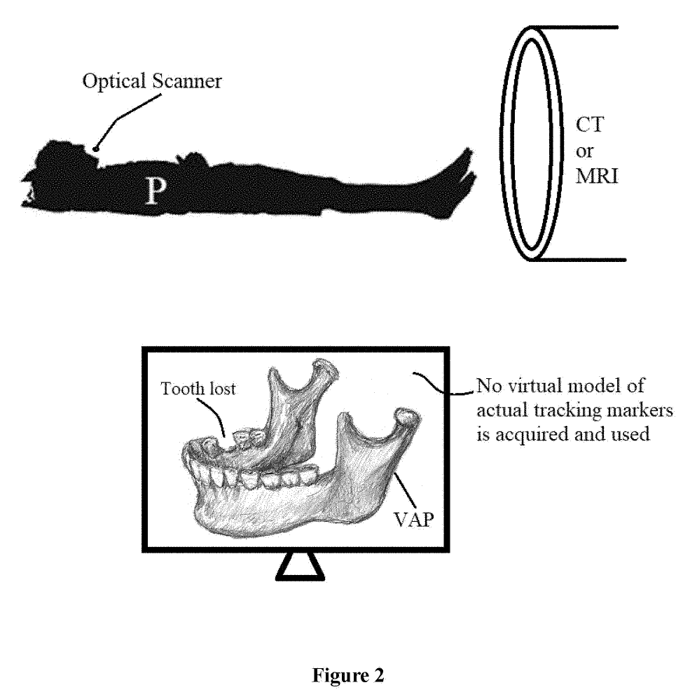

An embodiment of steps (1) and (2) is illustrated in FIG. 2. The actual anatomical part (AAP) is shown as mandibular jawbone and teeth of the patient P, with one lost tooth. There is no actual tracking marker (ATM) attached to the actual anatomical part in step (2). Even if there is, there will be no virtual model of the actual tracking markers (ATMs) is acquired in step (2) and subsequently used in any other step of the method. In preferred embodiments, the virtual anatomical part (VAP) includes an optical scan of the actual anatomical part (AAP) obtained from any known optical 3D scanner, such as an intraoral optical scanner, using for example multi-modality registration methods.

Referring back to FIG. 1, step (3) is attaching actual tracking markers (ATMs) to the actual anatomical part (AAP). As shown in FIG. 3, an actual tracking device 31 may be firmly clipped onto the patient P's actual anatomical part (AAP) such as a few healthy and strong teeth that do not shake, so that the actual anatomical part (AAP) (e.g. jawbone and teeth) and the actual tracking markers (ATMs) attached therewith will maintain an unchanged or defined spatial relationship during the following image guided medical procedure. The actual tracking markers (ATMs) include at least three tracking markers that are not on a same straight line. The actual tracking markers may have a geometric shape or contain a material that can be recognized by a computer vision system. The markers can be of any kind, such as geometric shapes, geometry patterns, passive IR markers, or active markers emitting electromagnetic signals.

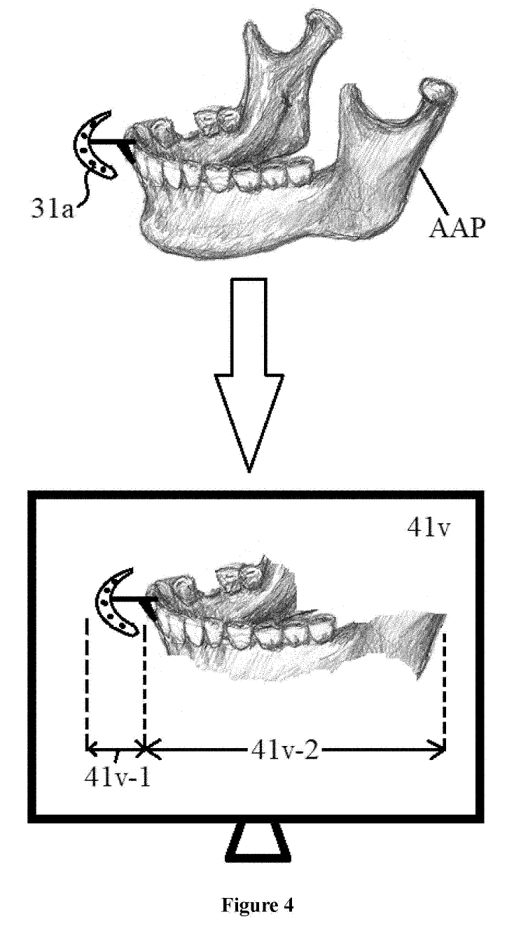

Referring back to FIG. 1, and as shown in FIG. 4, step (4) is acquiring a virtual combined model 41v of the actual tracking markers (ATMs) and at least a part of the actual anatomical part (AAP). The virtual combined model 41v comprises a first sub-model 41v-1 from, or based on, the actual tracking markers (ATMs) and a second sub-model 41v-2 from, or based on, the at least a part of the actual anatomical part (AAP).

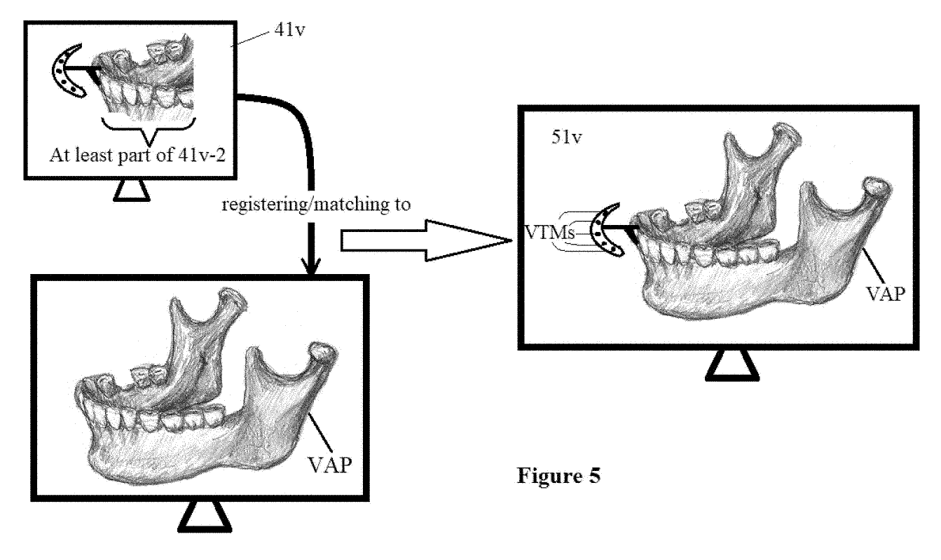

Referring back to FIG. 1, and as shown in FIG. 5, step (5) is registering the virtual combined model 41v to the virtual anatomical part (VAP) as obtained in step (2) by selecting at least a part of the second sub-model 41v-2 and matching the part to its counterpart in the virtual anatomical part (VAP). Step (6) is generating a working model 51v including the virtual anatomical part (VAP) and virtual tracking markers (VTMs). The VAP and the VTMs will have a spatial relationship that is the same as the spatial relationship in step (3) and as shown in FIG. 3.

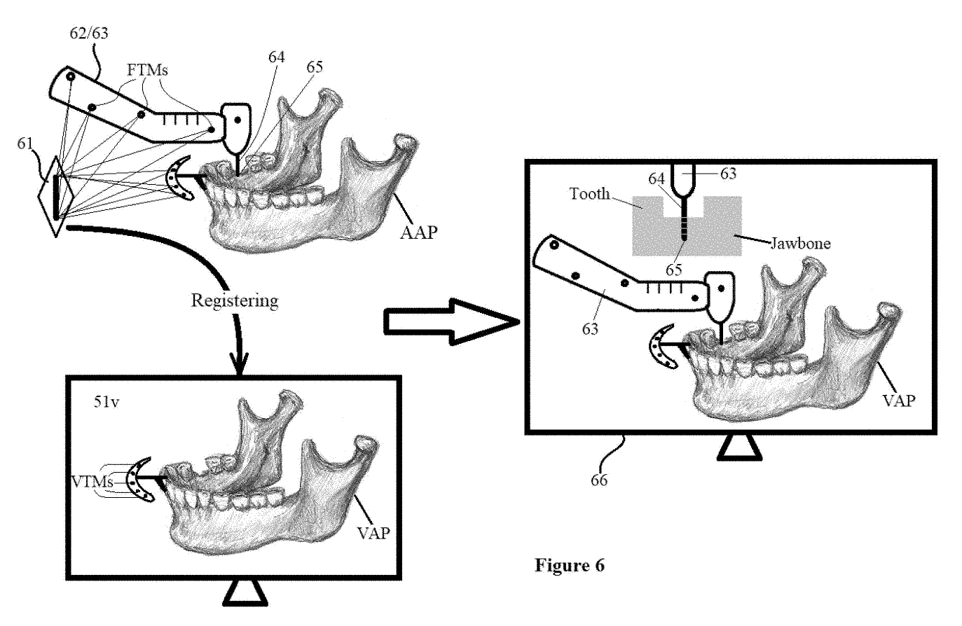

Referring back to FIG. 1, and as shown in FIG. 6, step (7) is, during the rest of the image guided medical procedure, tracking position and orientation of the actual tracking markers (ATMs) with a tracking device 61, registering the tracked position and orientation of the actual tracking markers (ATMs) to the working model 51v, and calculating and tracking position and orientation of the virtual anatomical part (VAP) in real-time based on the spatial relationship in step (6) which are the same as the position and orientation of the actual anatomical part (AAP).

Referring back to FIG. 1, and as shown in FIG. 6, the method of the invention may further comprise a step of (8) guiding movement of an object 62 such as a dental drill 63 that is foreign to the actual anatomical part (AAP). However, it is contemplated that object 62 can be any suitable instrument, tool, implant, medical device, delivery system, or any combination thereof. For example, object 62 can be a dental drill 63, a probe, a guide wire, an endoscope, a needle, a sensor, a stylet, a suction tube, a catheter, a balloon catheter, a lead, a stent, an insert, a capsule, a drug delivery system, a cell delivery system, a gene delivery system, an opening, an ablation tool, a biopsy window, a biopsy system, an arthroscopic system, or any combination thereof. In preferred embodiments, object 62 such as a dental handpiece (or a dental drill) 63 may also have at least 3 tracking markers (FTMs) that are not on a straight line. The drill bit 64 and drilling tip 65 have a known and defined spatial relationship relative to the at least 3 tracking markers (FTMs).

During the image guided medical procedure, position and orientation of the at least 3 tracking markers (FTMs) may be tracked with the same tracking device 61. The tracked position and orientation of the at least 3 tracking markers (FTMs) may then be registered to a pre-stored drill model with the known and defined spatial relationship between drill bit 64 with drilling tip 65 and the at least 3 tracking markers (FTMs). Therefore, position and orientation of the tracked (or virtual) drill bit 64 and drilling tip 65 may be calculated and tracked in real-time as their counterparts in reality are moving and/or rotating.

Because position and orientation of the actual drill bit 64, the actual drilling tip 65 and the actual anatomical part (AAP) such as jawbone and teeth are tracked under the same tracking device 61, and calculated in real-time as their counterparts in reality are moving and/or rotating, their 2D or 3D images will be overlapped, overlaid or superimposed. Therefore, the 3D images will enable a doctor to see the surgical details that his/her naked eyes cannot see. For example, when the actual dental drill 63 is partially drilled into the jawbone, the doctor will not be able to see, with his/her naked eyes, the part of actual drill bit 64 and drilling tip 65 that have been already “buried” into the jawbone. However, the doctor can see the overlapped, overlaid or superimposed 2D or 3D images as described above, which clearly demonstrate position and orientation of the part of actual drill bit 64 and drilling tip 65 that have been “buried” into the jawbone. Therefore, in preferred embodiments, the method, of the invention may further comprise a step of displaying in real-time the position and orientation of the actual anatomical part as tracked in step (6) in a displaying device such as computer monitor 66, as shown in FIG. 6.

### Two Specific Embodiments of Steps (4) and (5)

As described above, step (4) of the invention is “acquiring a virtual combined model of the actual tracking markers and at least a part of the actual anatomical part, wherein the virtual combined model comprises a first sub-model from the actual tracking markers and a second sub-model from the at least a part of the actual anatomical part”. Step (5) of the invention includes a specific sub-step of “selecting at least a part of the second sub-model and matching the part to its counterpart in the virtual anatomical part”. Referring back to FIG. 1 and FIG. 5, step (5) is registering the virtual combined model 41v to the virtual anatomical part (VAP) as obtained in step (2) by the specific sub-step X, i.e. selecting at least a part of the second sub-model 41v-2 and matching the part to its counterpart in the virtual anatomical part (VAP) Later, step (6) can be carried out to generate a working model 51v including the virtual anatomical part (VAP) and virtual tracking markers (VTMs). The VAP and the VTMs will have a spatial relationship that is the same as the spatial relationship in step (3) and as shown in FIG. 3.

### The First Specific Embodiment of Steps (4) and (5)

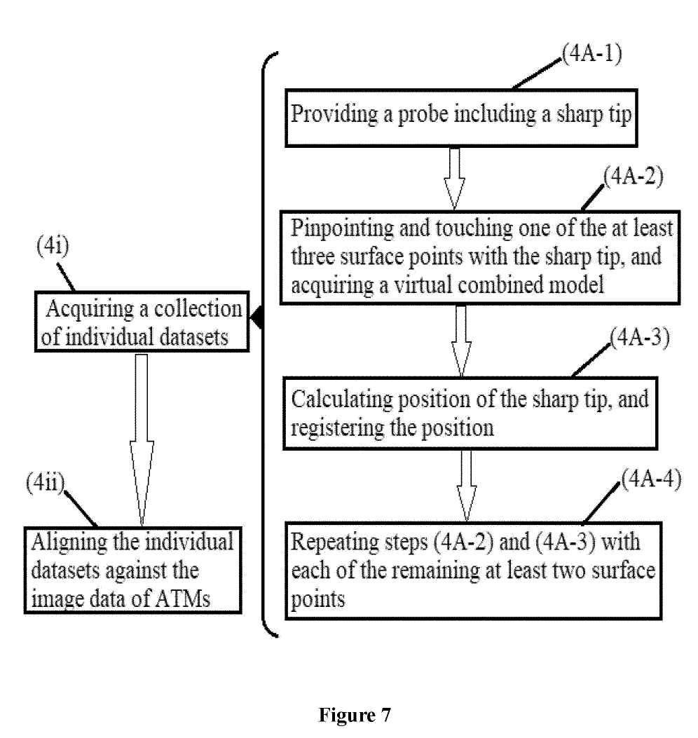

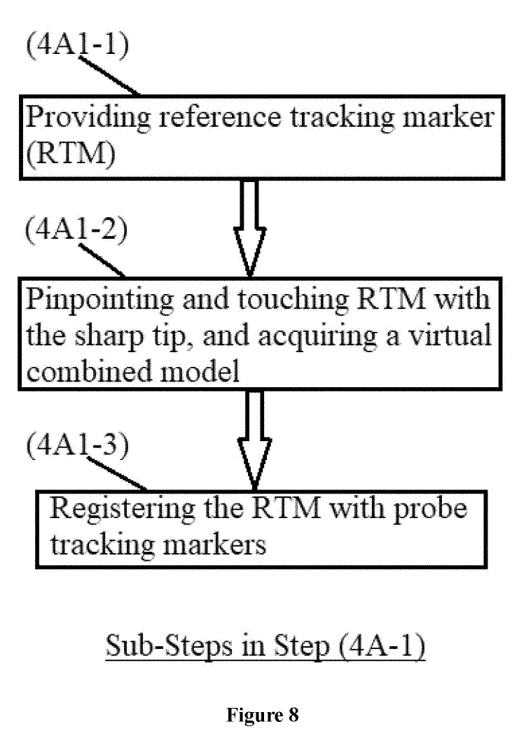

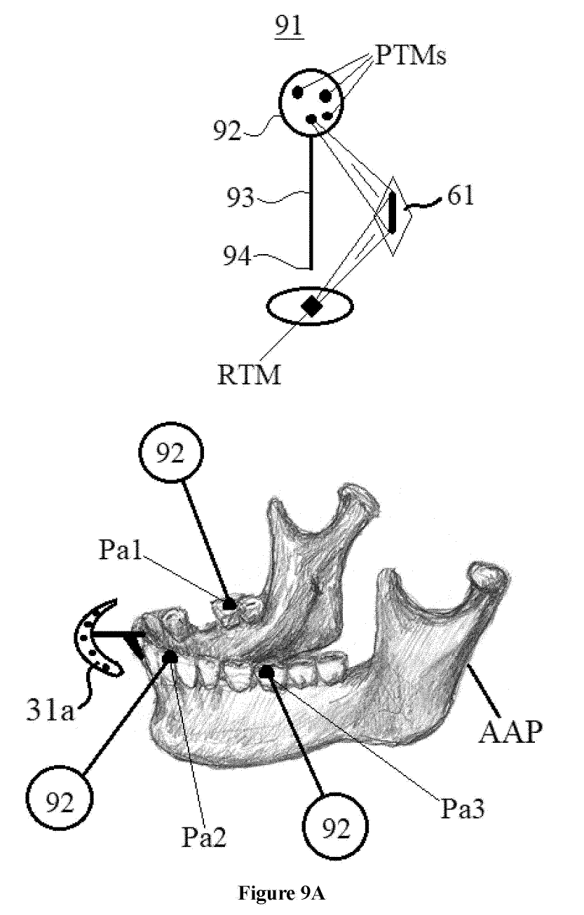

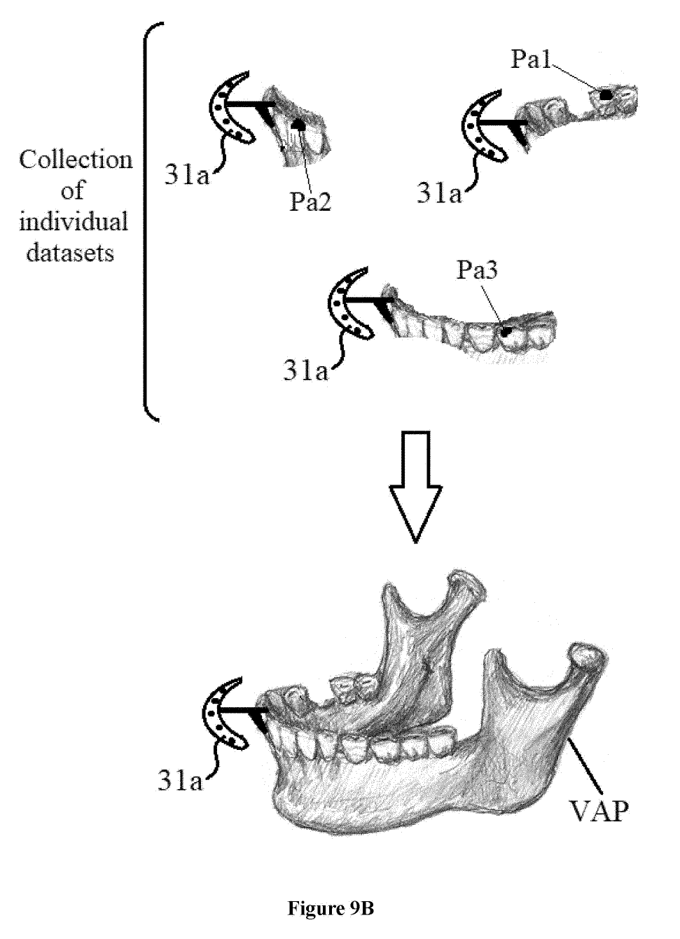

As shown in FIG. 7 and FIG. 9A, step (4A-1) is providing a probe 91 including a body 92 and an elongated member 93 extending from the body 92. The body has at least 3 probe tracking markers (PTMs) that are not on a same straight line, and the elongated member 93 has a sharp tip 94 that can be approximated as a geometrical point. The sharp tip 94 has a known and defined spatial relationship relative to the probe tracking markers (PTMs) The spatial relationship between the sharp tip 94 and the probe tracking markers (PTMs) may be pre-determined when the probe 91 is manufactured, or it may be determined using the sub-steps as shown in FIG. 8.

As shown in FIG. 8 and FIG. 9A, the spatial relationship between the sharp tip 94 and the probe tracking markers (PTMs) is acquired by the following sub-steps. Sub-step (4A1-1) is providing a reference tracking marker (RTM) Sub-step (4A1-2) is pinpointing and touching the reference tracking marker (RTM, e.g. a center thereof) with the sharp tip 94, and in the meanwhile, acquiring a virtual combined model of the reference tracking marker (RTM) and the probe tracking markers (PTMs), using for example tracking device 61. Sub-step (4A1-3) is registering the reference tracking marker (RTM) with the probe tracking markers (PTMs), which is treated as registering the sharp tip 94 with the probe tracking markers (PTMs), since the reference tracking marker (RTM) and the sharp tip 94 occupy the same geometrical point when step (4A1-2) is performed.

With the spatial relationship between the sharp tip 94 and the probe tracking markers (PTMs) being established, and referring back to FIG. 7, step (4A-2) is pinpointing and touching one (e.g. Pa1) of at least three surface points Pa1, Pa2 and Pa3 on the AAP such as teeth with the sharp tip 94, and in the meanwhile, acquiring a virtual combined model of (i) the probe tracking markers (PTMs) and (ii) the actual tracking markers (ATMs) that are attached to the actual anatomical part (AAP) There are at least three surface points Pv1, Pv2 and Pv3 (not shown) on the VAP, that are counterparts of surface points Pa1, Pa2 and Pa3 on'the AAP. At least three surface points Pa1, Pa2 and Pa3 may be for example 3 pinnacle points or apex points of 3 teeth.

Step (4A-3) is calculating the position of the sharp tip 94 from the probe tracking markers (PTMs) based on the spatial relationship therebetween that has been established in step (4A-1), registering the position of the sharp tip 94 with the tracking markers (VTMs) that are attached to the anatomical part in the virtual combined model, which is treated as registering the one of the at least three surface points (e.g. Pa1) with the tracking markers (VTMs) that are attached to the anatomical part in the virtual combined model, since surface point Pa1 and the sharp tip 94 occupy the same geometrical point when step (4A-2) is performed. As a result, an individual dataset that includes image data of the actual tracking markers and surface point Pa1 is obtained. Step (4A-4) is repeating steps (4A-2) and (4A-3) with each of the remaining at least two surface points (e.g. Pa2 and Pa3) to obtain their individual datasets and then, to complete the collection of the individual datasets, as shown in FIG. 9B.

Steps (4A-1)˜(4A-4) as described above constitute an exemplary embodiment of step (4i) as shown in FIG. 7. Step (4i) is acquiring a collection of individual datasets, wherein each of the individual dataset includes image data of the actual tracking markers (ATMs) and one of at least three surface points selected from the actual anatomical part (AAP), e.g. Pa1, Pa2 and Pa3

Referring back to FIG. 1, “acquiring a virtual combined model of the actual tracking markers and at least a part of the actual anatomical part” in step (4) is therefore carried out by above step (4i) followed by step (4ii), which is aligning the individual datasets against the image data of the actual tracking markers (ATMs). Now, the image data of the at least three surface points (Pa1, Pa2 and Pa3) after the aligning can represent the actual anatomical part (AAP)

As described above, step (5) of the invention includes a specific sub-step of “selecting at least a part of the second sub-model and matching the part to its counterpart in the virtual anatomical part” In this first specific embodiment, such specific sub-step is carried out by selecting at least three surface points (Pv1, Pv2 and Pv3, counterpart of Pa1, Pa2 and Pa3, not shown) from the second sub-model 41v-2 as shown in FIG. 4, and matching the at least three surface points to (Pv1, Pv2 and Pv3) their counterparts in the virtual anatomical part.

In a preferred embodiment, the probe 91 is dental drill 63, the elongated member 93 is drill bit 64, and the sharp tip 94 is the drilling tip 65 of the drill bit 64, as shown in FIGS. 6 and 9A.

Referring back to the best mode embodiment as described above, an optical scan of the patient is obtained through a model scanner or intra-oral scanner The scan is done as in normal dental CAD/CAM practice, and the resulted model has the patient's teeth and tissue surfaces. A procedure to accomplish the necessary registrations and the surgical process follows. 1— With the optical scan data, the implant treatment planning can be done now with the patient CT and optical scan data. A typical procedure will include loading the CT scan into the planning software, performing 3D reconstruction of the CT data, segment the tooth structures if necessary, load the optical scan into the system and register the two datasets with normal techniques such as ICP algorithm. 2— At the surgery time, the patient is in the field of view of the tracking cameras, and so is the surgical tool, i the hand piece. 3— The positioning device is now attached to the patient's teeth or tissue with enough distance from the implant site 4— A sharp tool, such as a drill with sharp tip or a needle is attached to the handpiece 5— A plate with additional tracking/fiducial markers is introduced into the view As an example of RTM as shown in FIG. 9A, this plate can also be just part of the positioning device 6— The doctor will place the tip of the sharp tool onto a predefined point of the plate, and at this moment, the computer software will record the geometric relationship between the tip of the sharp tool and the marker systems on the handpiece. In other words, the system can now always calculate the tip position of the sharp tool by tracking the hand piece. 7— Now in the computer software systems, a couple of points, for example three points, will be chosen on the model scan or intraoral scan. 8— Then the operator will use the needle to touch one point corresponding to the selected points of the patient's actual anatomy. 9— The system will then acquire the markers of the positioning device at this moment. 10— Then the doctor will continue touch the other points on the patient's anatomy corresponding to the earlier selected points This is just to repeat steps 8— and 9—. Every time, the touched point and the markers on the device will be obtained. 11— Register all the data acquired in steps 8— to 10— by registering the markers, the computer software will find out the three points on the patient jaw, and create a data set of the marker and the three points. 12— The computer will then register the three points with their counterparts on the model scan or intraoral scan obtained in step 9—, and therefore transfer the marker positions into the image data coordinate system. This way, the base for tracking the marker data is generated. 13— The spatial relationship between the markers on the positioning device and the actual patient anatomy is now worked out by transforming the marker positions together with the registered points in step 14. 14— From this point on, the system can completely track the patient's anatomy, the handpiece and surgical tools by tracking all the markers in the field of view, and the image guided surgery is performed as it is usually done.

Another embodiment may be that, when the optical scan is not obtained, the above workflow can be modified to initially pick points on the CT data and to pick their counterparts in actual patient's anatomy.

The Second Specific Embodiment of Steps (4) and (5)

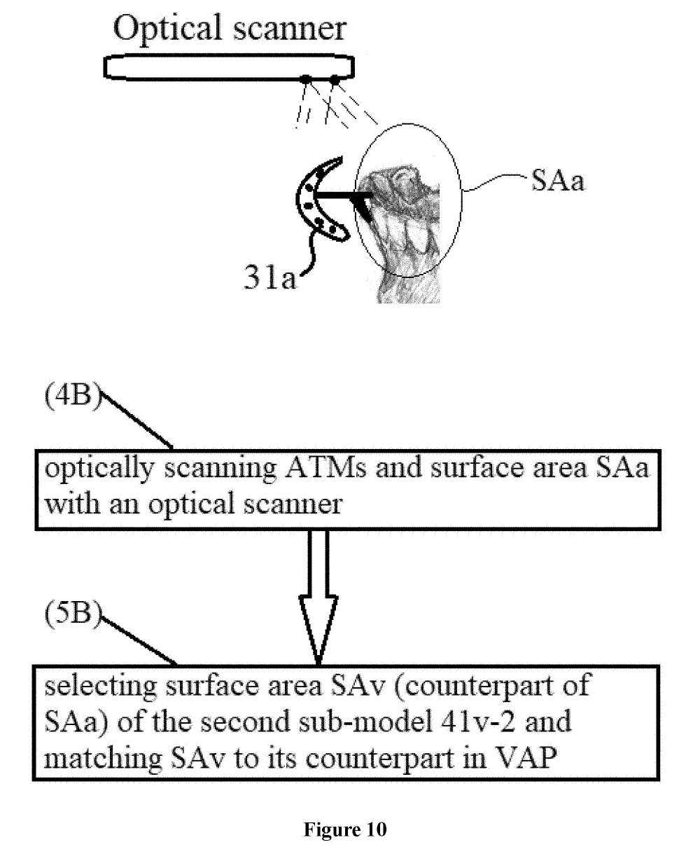

As described above, step (5) of the invention includes a specific sub-step of “selecting at least a part of the second sub-model and matching the part to its counterpart in the virtual anatomical part”. In this first specific embodiment, such specific sub-step is carried out by (5B)— selecting a surface area SAv (not shown) of the second sub-model (41v-2) and matching the surface area SAY to its counterpart in the virtual anatomical part (VAP) To accomplish the sub-step, “acquiring a virtual combined model of the actual tracking markers and at least a part of the actual anatomical part” in step (4) should be carried out first by, for example, (4B)— optically scanning the actual tracking markers (ATMs) and at least a part of the actual anatomical part's surface area (SAa, counterpart of SAv) with an optical scanner, as shown in FIG. 10. The virtual combined model so acquired is an optical scan dataset. For example, the optical scanning may be an intra oral scanning, and the virtual combined model so acquired is an intra oral scan dataset.

Referring back to the best mode embodiment as described above, an intra-oral scan may be obtained with some modifications. (1)— Either before or after the CT scan, a positioning device is attached onto patient's anatomy, typically, one or more teeth. It does not matter how the geometry will be and how it is attached, but as long as it is attached, (2)— An intra-oral scan is then performed. The scan will be extended to the positioning device and the markers on the device. (3)— After the intra-oral scan is loaded into the software and register with patient's CT data, the system will identify the tracking/fiducial markers on the positioning device portion of the intra-oral scan. This can be either automatically performed, or manually specified. At this point of time, the computer software system has the complete information for image based navigation: patient CT data, optical san, and the fiducial markers. (4)— The surgical handpiece and drills can now be registered with the patient data by the tracking device and corresponding software module. (5)— The tracking device is then continuously tracking the positions of the markers so as to calculate the actual patient position, and tracking the relative positions between the markers and the surgical tools, and thus provides image rand graphical feedback for the doctor to continue the surgery guided by the tracking data and image data.

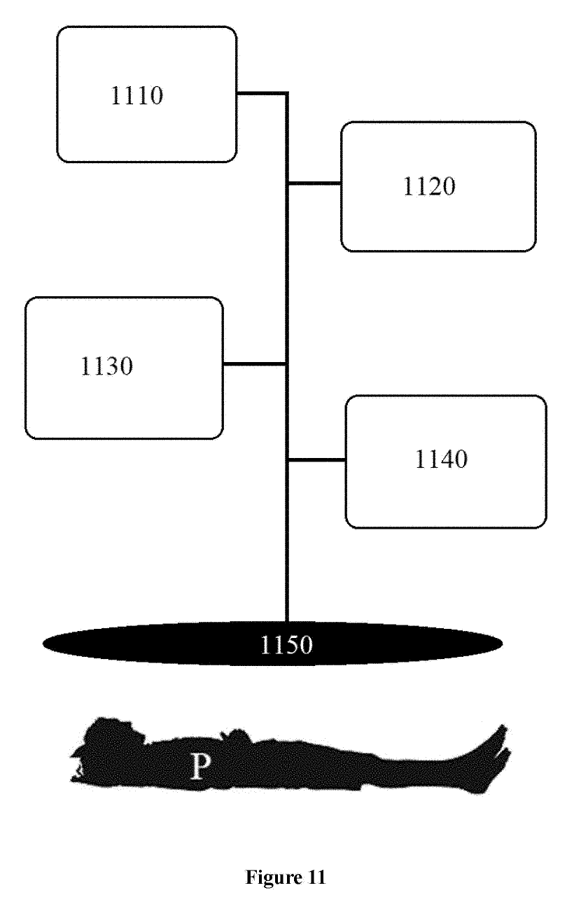

Another aspect of the invention provides an apparatus for performing the image guided medical procedure as described above. As shown in FIG. 11, the apparatus includes a first module (or control circuit) 1110 for generating, a virtual anatomical part for treatment planning from at least CT or MRI scanning of an actual anatomical part of a patient.

The apparatus includes a second module (or control circuit) 1120 for acquiring a virtual combined model of actual tracking markers and at least a part of an actual anatomical part, wherein the virtual combined model comprises a first sub-model from the actual tracking markers and a second sub-model from the at least a part of the actual anatomical part; wherein the actual tracking markers are attached to the actual anatomical part; and wherein the actual anatomical part and the actual tracking markers attached therewith maintain a spatial relationship during the image guided medical procedure.

The apparatus includes a third module (or control circuit) 1130 for registering the virtual combined model to the virtual anatomical part by selecting at least a part of the second sub-model and matching the part to its counterpart in the virtual anatomical part,

The apparatus includes a fourth module (or control circuit) 1140 for generating a working model including the virtual anatomical part and virtual tracking markers, the two having a spatial relationship same as the spatial relationship in the second module.

The apparatus includes a tracking system 1150 for tracking position and orientation of the actual tracking markers, registering the tracked position and orientation of the actual tracking markers to the working model, and calculating and tracking position and orientation of the virtual anatomical part in real-time based on the spatial relationship in the fourth module, which are the same as the position and orientation of the actual anatomical part, during the image guided medical procedure.

In preferred embodiments of the first module 1110, there is no actual tracking marker attached to the actual anatomical part, or there is no virtual model of actual tracking markers is acquired and subsequently used in any other step of, when at least CT or MRI is scanning the actual anatomical part.

In some embodiments of the third module 1130, the “selecting at least a part of the second sub-model and matching the part to its counterpart in the virtual anatomical part” is carried out by selecting at least three surface points from the second sub-model and matching the at least three surface points to their counterparts in the virtual anatomical part. Accordingly, in the second module, the “acquiring a virtual combined model of the actual tracking markers and at least a part of the actual anatomical part” is carried out by (4i) acquiring a collection of individual datasets, wherein each of the individual dataset includes image data of the actual tracking markers and one of the at least three surface points, and (4ii) aligning the individual datasets against the image data of the actual tracking markers, wherein image data of the at least three surface points after the aligning can represent the actual anatomical part.

Step (4i) may be carried out by (4A-1) providing a probe including a body and an elongated member extending from the body, wherein the body has probe tracking markers, wherein the elongated member has a sharp tip that can be approximated as a geometrical point, and wherein the sharp tip has a defined spatial relationship relative to the probe tracking markers; (4A-2) pinpointing and touching one of the at least three surface points with the sharp tip, and in the meanwhile, acquiring a virtual combined model of (i) the probe tracking markers and (ii) the actual tracking markers that are attached to the actual anatomical part; (4A-3) calculating position of the sharp tip from the probe tracking markers based on the spatial relationship therebetween, registering the position of the sharp tip with the tracking markers that are attached to the anatomical part in the virtual combined model, which is treated as registering the one of the at least three surface points with the tracking markers that are attached to the anatomical part in the virtual combined model, since the one of the at least three surface points and the sharp tip occupy the same geometrical point when step (4A-2) is performed, so as to obtain an individual dataset that includes image data of the actual tracking markers and the one of the at least three surface points; and (4A-4) repeating steps (4A-2) and (4A-3) with each of the remaining at least two surface points, until all individual datasets are obtained to complete the collection of the individual datasets. The defined spatial relationship between the sharp tip and the probe tracking markers may be acquired by (4A1-1) providing a reference tracking marker; (4A1-2) pinpointing and touching the reference tracking marker (e.g. a center thereof) with the sharp tip, and in the meanwhile, acquiring a virtual combined model of the reference tracking marker and the probe tracking markers; and (4A1-3) registering the reference tracking marker with the probe tracking markers, which is treated as registering the sharp tip with the probe tracking markers, since the reference tracking marker and the sharp tip occupy the same geometrical point when step (4A1-2) is performed.

In other embodiments of the third module 1130, the “selecting at least a part of the second sub-model and matching the part to its counterpart in the virtual anatomical part” is carried out by (5B) selecting a surface area of the second sub-model and matching the surface area to its counterpart in the virtual anatomical part. Accordingly, in the second module, the “acquiring a virtual combined model of the actual tracking markers and at least a part of the actual anatomical part” is carried out by (4B) optically scanning the actual tracking markers and at least a part of the actual anatomical part's surface area, and the virtual combined model so acquired is an optical scan dataset.

Referring back to the best mode embodiment as described above, a system for the embodiment may include one or more of the following components: a tracking system, a computer system with memory and CPU etc., a surgical handpiece with tracking markers, a positioning device such as a clip with tracking markers, a treatment planning software module, a treatment preparation module, and a treatment execution module The treatment preparation module registers the patient's anatomy with the pre-op treatment plan, and registers the handpiece and the tip of drilling or probing tools. The preparation module has the following functional components: a— Tool registration: Register the tool tip with the handpiece; b— Device/Point (e.g. Clip/Point) Registration. Patient anatomical point acquisition and register with the markers on the clip; and c— Device/Patient (e.g. Clip/Point) Registration: combine at least three pairs of Clip/Point registration data to get a Device/Patient registration result, rand register Clip/Patient with pre-op data. The treatment execution module is for tracking and displaying the tool positions with respect to the patient positions; and tracking and displaying the tool positions with respect to the planned implant positions.

As a reader can appreciate, techniques and technologies may be described herein in terms of functional and/or logical block components and with reference to symbolic representations of operations, processing tasks, and functions that may be performed by various computing components or devices. Such operations, tasks, and functions are sometimes referred to as being computer-executed, computerized, processor-executed, software-implemented, or computer-implemented. It should be appreciated that the various block components shown in the figures may be realized by any number of hardware, software, and/or firmware components configured to perform the specified functions For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or executable instructions that, when executed by one or more processor devices, cause the host computing system to perform the various tasks. In certain embodiments, the program or code segments are stored in a tangible processor-readable medium, which may include any medium that can store or transfer information Examples of suitable forms of non-transitory and processor-readable media include an electronic circuit, a semiconductor memory device, a ROM, a flash memory, an erasable ROM (EROM), a floppy diskette, a CD-ROM, ran optical disk, a hard disk, or the like.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. A method of performing an image guided medical procedure, comprising
(1) providing an actual anatomical part of a patient,
(2) generating, a virtual anatomical part for treatment planning from at least CT or MIRE scanning of the actual anatomical part;
(3) attaching actual tracking markers to the actual anatomical part, wherein the actual anatomical part and the actual tracking markers attached therewith maintain a spatial relationship during the image guided medical procedure;
(4) acquiring a virtual combined model of the actual tracking markers and at least a part of the actual anatomical part, wherein the virtual combined model comprises a first sub-model from the actual tracking markers and a second sub-model from said at least a part of the actual anatomical part;
(5) registering the virtual combined model to said virtual anatomical part by selecting at least a part of the second sub-model and matching the part to its counterpart in the virtual anatomical part,
(6) generating a working model including the virtual anatomical part and virtual tracking markers, the two having a spatial relationship same as the spatial relationship in step (3), and
(7) during the rest of the image guided medical procedure, tracking position and orientation of the actual tracking markers, registering the tracked position and orientation of the actual tracking, markers to the working model, and calculating and tracking position and orientation of the virtual anatomical part in real-time based on the spatial relationship in step (6) which are the same as the position and orientation of the actual anatomical part.

2. The method according to claim 1, wherein there is no actual tracking marker attached to the actual anatomical part in step (2).

3. The method according to claim 1, wherein there is no virtual model of actual tracking markers is acquired in step (2) and subsequently used in any other step of the method.

4. The method according to claim 1, wherein said virtual anatomical part further comprises an optical scan of the actual anatomical part.

5. The method according to claim 1, wherein the actual anatomical part includes teeth and jawbone of the patient.

6. The method according to claim 1, wherein the actual tracking markers include at least three tracking markers that are not on a same straight line.

7. The method according to claim 1, wherein “selecting at least a part of the second sub-model and matching the part to its counterpart in the virtual anatomical part” in step (5) is carried out by selecting at least three surface points from the second sub-model and matching the at least three surface points to their counterparts in the virtual anatomical part.

8. The method according to claim 7, wherein “acquiring a virtual combined model of the actual tracking markers and at least a part of the actual anatomical part” in step (4) is carried out by (4i) acquiring a collection of individual datasets, wherein each of the individual dataset includes image data of the actual tracking markers and one of the at least three surface points; and (4ii) aligning the individual datasets against the image data of the actual tracking markers, wherein image data of the at least three surface points after the aligning can represent the actual anatomical part.

9. The method according to claim 8, wherein step (4i) is carried out by
(4A-1) providing a probe including a body and an elongated member extending from the body, wherein the body has probe tracking markers, wherein the elongated member has a sharp tip that can be approximated as a geometrical point, and wherein the sharp tip has a defined spatial relationship relative to the probe tracking markers;
(4A-2) pinpointing and touching one of the at least three surface points with the sharp tip, and in the meanwhile, acquiring a virtual combined model of (i) the probe tracking markers and (ii) the actual tracking markers that are attached to the actual anatomical part,
(4A-3) calculating position of the sharp tip from the probe tracking markers based on the spatial relationship therebetween, registering the position of the sharp tip with the tracking markers that are attached to the anatomical part in the virtual combined model, which is treated as registering said one of the at least three surface points with the tracking markers that are attached to the anatomical part in the virtual combined model, since said one of the at least three surface points and the sharp tip occupy the same geometrical point when step (4A-2) is performed, so as to obtain an individual dataset that includes image data of the actual tracking markers and said one of the at least three surface points,
(4A-4) repeating steps (4A-2) and (4A-3) with each of the remaining at least two surface points, until all individual datasets are obtained to complete the collection of the individual datasets.

10. The method according to claim 9, wherein the defined spatial relationship between the sharp tip and the probe tracking markers is acquired by
(4A1-1) providing a reference tracking marker;
(4A1-2) pinpointing and touching the reference tracking marker (e.g. a center thereof) with the sharp tip, and in the meanwhile, acquiring a virtual combined model of the reference tracking marker and the probe tracking markers; and
(4A1-3) registering the reference tracking marker with the probe tracking markers, which is treated as registering the sharp tip with the probe tracking markers, since the reference tracking marker and the sharp tip occupy the same geometrical point when step (4A1-2) is performed.

11. The method according to claim 9, wherein the probe is a dental drill, the elongated member is a drill bit, and the sharp tip is the drilling tip of the drill bit.

12. The method according to claim 1, wherein “selecting at least a part of the second sub-model and matching the part to its counterpart in the virtual anatomical part” in step (5) is carried out by (5B) selecting a surface area of the second sub-model and matching the surface area to its counterpart in the virtual anatomical part.

13. The method according to claim 12, wherein “acquiring a virtual combined model of the actual tracking markers and at least a part of the actual anatomical part” in step (4) is carried out by (4B) optically scanning the actual tracking markers and at least a part of the actual anatomical part's surface area, and the virtual combined model so acquired is an optical scan dataset.

14. The method according to claim 13, wherein the optical scanning is an intra oral scanning, and the virtual combined model so acquired is an intra oral scan dataset,

15. The method according to claim 1, further comprising displaying in real-time the position and orientation of the actual anatomical part as tracked in step (6).

16. The method according to claim 1, wherein the actual tracking markers in step (3) have a geometric shape or contain a material that can be recognized by a computer vision system.

17. The method according to claim 1, further comprising guiding movement of an object foreign to the actual anatomical part.

18. The method according to claim 17, wherein the object foreign to the actual anatomical part is an instrument, a tool, an implant, a medical device, a delivery system, or any combination thereof.

19. The method according to claim 17, wherein the object foreign to the actual anatomical part is a dental drill, a probe, a guide wire, an endoscope, a needle, a sensor, a stylet, a suction tube, a catheter, a balloon catheter, a lead, a stent, an insert, a capsule, a drug delivery system, a cell delivery system, a gene delivery system, an opening, an ablation tool, a biopsy window, a biopsy system, an arthroscopic system, or any combination thereof.

20. An apparatus for performing an image guided medical procedure, comprising
(1) a first module (or control circuit) for generating a virtual anatomical part for treatment planning from at least CT or MRI scanning of an actual anatomical part of a patient;
(2) a second module (or control circuit) for acquiring a virtual combined model of actual tracking markers and at least a part of an actual anatomical part, wherein the virtual combined model comprises a first sub-model from the actual tracking markers and a second sub-model from said at least a part of the actual anatomical part; wherein the actual tracking markers are attached to the actual anatomical part; and wherein the actual anatomical part and the actual tracking markers attached therewith maintain a spatial relationship during the image guided medical procedure;
(3) a third module (or control circuit) for registering the virtual combined model to said virtual anatomical part by selecting at least a part of the second sub-model and matching the part to its counterpart in the virtual anatomical part;
(4) a fourth module (or control circuit) for generating a working model including the virtual anatomical part and virtual tracking markers, the two having a spatial relationship same as the spatial relationship in the second module; and
(5) a tracking system for tracking position and orientation of the actual tracking markers, registering the tracked position and orientation of the actual tracking markers to the working, model, and calculating and tracking position and orientation of the virtual anatomical part in real-time based on the spatial relationship in the fourth module, which are the same as the position and orientation of the actual anatomical part, during the image guided medical procedure.

21. The apparatus according to claim 20, wherein there is no actual tracking marker attached to the actual anatomical part, or there is no virtual model of actual tracking markers is acquired and subsequently used in any other step of, when at least CT or MRI is scanning the actual anatomical part.

22. The apparatus according to claim 20, wherein, in the third module, said “selecting, at least a part of the second sub-model and matching the part to its counterpart in the virtual anatomical part” is carried out by selecting at least three surface points from the second sub-model and matching the at least three surface points to their counterparts in the virtual anatomical part.

23. The apparatus according to claim 22, wherein, in the second module, said “acquiring a virtual combined model of the actual tracking markers and at least a part of the actual anatomical part” is carried out by (4i) acquiring a collection of individual datasets, wherein each of the individual dataset includes image data of the actual tracking markers and one of the at least three surface points; and (4ii) aligning the individual datasets against the image data of the actual tracking markers, wherein image data of the at least three surface points after the aligning can represent the actual anatomical part.

24. The apparatus according to claim 23, wherein step (4i) is carried out by
(4A-1) providing a probe including a body and an elongated member extending from the body, wherein the body has probe tracking markers, wherein the elongated member has a sharp tip that can be approximated as a geometrical point, and wherein the sharp tip has a defined spatial relationship relative to the probe tracking markers;
(4A-2) pinpointing and touching one of the at least three surface points with the sharp tip, and in the meanwhile, acquiring a virtual combined model of (i) the probe tracking markers and (ii) the actual tracking markers that are attached to the actual anatomical part;
(4A-3) calculating position of the sharp tip from the probe tracking markers based on the spatial relationship therebetween, registering the position of the sharp tip with the tracking markers that are attached to the anatomical part in the virtual combined model, which is treated as registering said one of the at least three surface points with the tracking markers that are attached to the anatomical part in the virtual combined model, since said one of the at least three surface points and the sharp tip occupy the same geometrical point when step (4A-2) is performed, so as to obtain an individual dataset that includes image data of the actual tracking, markers and said one of the at least three surface points; and
(4A-4) repeating steps (4A-2) and (4A-3) with each of the remaining at least two surface points, until all individual datasets are obtained to complete the collection of the individual datasets

25. The apparatus according to claim 24, wherein the defined spatial relationship between the sharp tip and the probe tracking markers is acquired by
(4A1-1) providing a reference tracking marker;
(4A1-2) pinpointing and touching the reference tracking marker (e.g. a center thereof) with the sharp tip, and in the meanwhile, acquiring a virtual combined model of the reference tracking marker and the probe tracking markers; and
(4A1-3) registering the reference tracking marker with the probe tracking markers, which is treated as registering the sharp tip with the probe tracking markers, since the reference tracking marker and the sharp tip occupy the same geometrical point when step (4A1-2) is performed

26. The apparatus according to claim 20, wherein, in the third module, said “selecting, at least a part of the second sub-model and matching the part to its counterpart in the virtual anatomical part” is carried out by (5B) selecting a surface area of the second sub-model and matching the surface area to its counterpart in the virtual anatomical part.

27. The apparatus according to claim 26, wherein, in the second module, said “acquiring a virtual combined model of the actual tracking markers and at least a part of the actual anatomical part” is carried out by (4B) optically scanning the actual tracking markers and at least a part of the actual anatomical part's surface area, and the virtual combined model so acquired is an optical scan dataset.
